(19) United States
(12) Patent Application Publication
(10) Pub. No.: US 2025/0380656 A1
(43) Pub. Date: Dec. 18, 2025

(54) APPLICATION OF ZM00001D030087 GENE IN REGULATING PROTEIN CONTENT OF MAIZE KERNELS

(71) Applicant: Institute of Food Crops, Yunnan Academy of Agricultural Sciences, Kunming (CN)

(72) Inventors: Xingming Fan, Kunming (CN); Xiaoping Yang, Kunming, Yunnan (CN); Fuyan Jiang, Kunming (CN)

(21) Appl. No.: 19/193,692

(22) Filed: Apr. 29, 2025

(30) Foreign Application Priority Data
Jun. 18, 2024 (CN) 202410782283.3

(51) Int. Cl. A01H 1/04; A01H 1/00

(52) U.S. Cl. CPC A01H 1/045; A01H 1/108

(57) ABSTRACT

The disclosure relates to the field of agricultural biotechnologies, and an application of a Zm00001d030087 gene in regulating protein content of maize kernels is provided. A sequence of the Zm00001d030087 gene is as shown in SEQ ID NO: 1. The gene is identified as a functional gene for regulating kernel protein content. By the gene, the protein content of the maize kernels can be regulated, and a basis for breeding maize varieties with high protein content can be provided.

### CROSS-REFERENCE TO RELATED APPLICATION

This application claims priority of Chinese Patent Application No. 202410782283.3, filed on Jun. 18, 2024, the entire contents of which are incorporated herein by reference.

### REFERENCE TO A SEQUENCE LISTING SUBMITTED VIA EFS-WEB

The content of the xml file of the sequence listing named “HKIP-US-1-1353-22_sequence_listing” which is 7,872 b in size was created on Apr. 18, 2025 and electronically submitted via EFS_Web herewith. These sequence listing is incorporated herein by reference in its entirety.

### TECHNICAL FIELD

The disclosure relates to the field of agricultural biotechnologies, and specifically relates to an application of a Zm00001d030087 gene in regulating protein content of maize kernels.

### BACKGROUND

Protein is one of the main nutrient substances stored in maize kernels. Maize protein, except for maintaining normal physiological functions of human bodies, is extensively applied to the fields of food processing and livestock breeding. By solubility, the protein in maize kernels can be classified into prolamines, albumins, globulins, and glutelins (Huang et al., 2022). Most maize has relatively poor quality due to a larger account of prolamines in endosperm and a small account of glutelins. In the context of challenges of a growing population and environmental dramatic changes globally, breeders have been working to improve maize, so as to enhance its nutritive values and its effectiveness as food and feed. Additionally, with the continuous progress of society and the improvement of human living standard, maize quality is increasingly focused on. Accordingly, as an important indicator for measuring maize quality, the protein content is widely concerned. Currently, maize is mainly used for feed production. However, the relatively low protein content of maize kernels fails to satisfy the daily nutrient and growth demand of animals, and additionally added high protein products such as soybean are required to make up for this deficiency. Since China is heavily dependent on soybean imports, discovering genes related to increasing protein content of maize kernels and developing maize varieties with high protein content have become important directions in maize genetic research.

Tropical and subtropical maize germplasms contain abundant genetic variation that many temperate maize lacks and serve as vital germplasm resources for maize breeding. In this study, a multiparent population (MPP) with rich variation in kernel protein content is constructed using five tropical and subtropical maize inbred lines with significant differences in kernel protein content. All the five materials used as parents are inbred lines which have important breeding values (Yin et al., 2022; Jiang et al., 2023). Exploring functional genes closely related to protein content of maize kernels from the MPP composed of the five parents provides a theoretical basis for molecular marker-assisted selection of maize with high protein content.

### SUMMARY

In view of the shortcomings in the prior art, the disclosure provides an application of a Zm00001d030087 gene in regulating protein content of maize kernels. The functional gene Zm00001d030087, which is closely associated with the protein content of maize kernels, is discovered, providing a theoretical basis for molecular marker-assisted selection of maize with high protein content.

To realize the above objective, the disclosure employs the following technical solutions.

An application of a Zm00001d030087 gene in regulating protein content of maize kernels is provided, and a sequence of the Zm00001d030087 gene is as shown in SEQ ID NO: 1.

An application of a Zm00001d030087 protein in regulating protein content of maize kernels is provided, and the Zm00001d030087 protein is obtained by encoding the gene which has the sequence as shown in SEQ ID NO: 1.

A product is provided, and the product contains a substance that regulates the Zm00001d030087 gene which has the sequence as shown in SEQ ID NO: 1.

Preferably, the product is a kit.

The product can be applied to molecular mark-assisted breeding of gramineous crops or improvement of agronomic traits of the gramineous crops.

Preferably, the agronomic traits include: changing protein content of the gramineous crops, changing kernels protein content of the gramineous crops or improving quality of the gramineous crops.

Preferably, the gramineous crops are maize.

In addition, the disclosure further provides a maize breeding method for increasing kernel protein content: manipulating the expression of the Zm00001d030087 gene or protein in overexpressed maize, the sequence of the Zm00001d030087 gene being as shown in SEQ ID NO: 1.

A maize breeding method for decreasing kernel protein content is provided: silencing, knocking out or knocking down the Zm00001d030087 gene or protein in maize, the sequence of the Zm00001d030087 gene being as shown in SEQ ID NO: 1. In practical application, the demand for maize quality traits is not uniform, so it is also possible to reduce the protein content of maize kernels to obtain maize with high starch and high oil content, which is also one of the demands.

The disclosure provides an application of the Zm00001d030087 gene in regulating protein content of maize kernels, which has the following advantages over the prior art:

In this study, a temperate maize inbred line Ye107 with relatively low kernel protein content is used as a common parent, which is crossed with four subtropical maize inbred lines having relatively high kernel protein content, to construct a maize MPP which has significant differences in kernel protein content. Genome-wide association study (GWAS) analysis and genetic linkage analysis are utilized to co-localize SNP 104665306 which is on chromosome 1 and significantly associated with kernel protein content, and the functional gene Zm00001d030087 that regulates kernel protein content is further discovered. The gene Zm00001d030087 can explain 10.06% of the phenotypic variation in kernel protein content. Haplotype analysis indicates that in 601 recombinant inbred lines (RILs), Zm00001d030087 has two haplotypes (i.e., Hap1 and Hap2), and the kernel protein content of Hap2 is significantly higher than that of Hap1. Therefore, Hap2 of the Zm00001d030087 gene is a haplotype type that significantly increases kernel protein content. The results of this study favor for further research on a regulatory mechanism of protein content of maize kernels, and also provide a theoretical basis for developing maize varieties with high protein content.

### BRIEF DESCRIPTION OF THE DRAWINGS

FIG. 1a shows principal component analysis (PCA) for a population structure of 601 RILs in an embodiment of the disclosure;

FIG. 1b shows an unrooted tree for the population structure of 601 RILs in the embodiment of the disclosure;

FIG. 1c shows a phylogenetic tree for the population structure of 601 RILs in the embodiment of the disclosure; and

FIG. 1d shows a Bayesian clustering plot of the 601 RILs when K=4.

FIG. 2 shows a linkage disequilibrium (LD) decay plot of the 601 RILs in the embodiment of the disclosure.

FIG. 3 shows a genetic linkage map of pop2 in the embodiment of the disclosure.

FIG. 4 shows significant quantitative trait loci (QTLs) of protein identified in pop2 at Yanshan (YS) in the embodiment of the disclosure.

FIG. 5 shows significant QTLs of protein identified in pop2 at Jinghong (JH) in the embodiment of the disclosure.

FIG. 6a shows a Manhattan plot based on a protein phenotypic mean at YS in the embodiment of the disclosure;

FIG. 6b shows a quantile-quantile (QQ) plot based on the protein phenotypic mean at YS in the embodiment of the disclosure;

FIG. 6c shows a Manhattan plot based on a protein phenotypic mean at JH in the embodiment of the disclosure;

FIG. 6d shows a QQ plot based on the protein phenotypic mean at JH in the embodiment of the disclosure;

FIG. 6e shows a Manhattan plot based on a best linear unbiased prediction (BLUP) value for protein in the embodiment of the disclosure; and

FIG. 6f shows a QQ plot based on the BLUP value for protein in the embodiment of the disclosure.

FIG. 7a shows the haplotype analysis of protein candidate genes in the embodiment of the disclosure; and

FIG. 7b shows the distribution of haplotypes of Zm00001d030087 in four subpopulations.

### DETAILED DESCRIPTION

For clearer objective, technical solutions and advantages of the disclosure, the technical solutions of the embodiment in the disclosure will be described clearly and completely by reference to the accompanying drawings of the embodiment in the disclosure below. Obviously, the embodiment described is only some, rather than all embodiments of the disclosure. On the basis of the embodiment of the disclosure, all other embodiments obtained by those ordinary skilled in the art without creative efforts are included in the scope of protection of the disclosure.

### Embodiment 1

### 1. Experiment

### 1.1 Plant Materials and Field Trials

To ensure a rich diversity of trial materials, in this study, the temperature excellent maize inbred line Ye107, and tropical and subtropical backbone maize inbred lines CML384, CML395, YML46 and YML32 were selected as parents. The five parents were derived from Reid, non-Reid and Suwan1 heterosis groups.

YS (23°19′−23°59′N, 103°35′-104°45′E) and JH (21°27′−22°36′N, 100°25′-101°31′E) of Yunnan province in China were selected as trial sites.

Ye107 was used as a common male parent, which was separately crossed with CML384, CML395, YML46 and YML32, to breed 4 hybrids (F1). After nine generations of single cross and self-cross, an MPP consisting of four subpopulations (pop1: CML384×Ye107; pop2: CML395×Ye107; pop3: YML46×Ye107; and pop4: YML32×Ye107) was generated. The MPP included 601 RIL families, with pop1, pop2, pop3 and pop4 having 161, 123, 145 and 172 families, respectively. Pedigree, ecological type, and protein content of the five parental lines are listed in Table 1 below.

TABLE 1
Parental information

| | | Ecological | Protein |
|---|---|---|---|
| Parents | Pedigree | type | content (%) |
| Ye107 | Derived from US hybrid DeKalb | Temperate | 10.1 |
| | XL80 | | |
| CML384 | P502c1#-771-2-2-1-3-B-1-1-3- | Subtropical | 7.7 |
| | 1(DH) | | |
| CML395 | 90323B-1-B-1-B*4-1-1-2-1(DH) | Tropical | 9.7 |
| YML46 | SW1-1-1-2-1-2-1 | Tropical | 11.9 |
| YML32 | Suwan 1(S)C9-S8-346-2 (Kei | Tropical | 10.2 |
| | 8902)-3-4-4-6 | | |

A completely randomized block design was employed in the experiment, with three replicates for each site. A field trial plot was 3 meters long, with a row spacing of 0.70 meters, 14 plants for each row, and two rows for each plot. The trials were conducted at YS and JH in year of 2022 and 2023.

### 1.2 Data Collection and Analysis

Kernels of 601 RILs were determined for protein content by using near infrared reflectance spectroscopy (NIRS, No. S-14105 Kungens Kurva, Sweden), with three replicates for each parent line, and a mean was used as a final value. Using IBM SPSS 26.0 software, phenotypic data, including mean, standard deviation, skewness, kurtosis, range of variation and coefficient of variation, were subjected to statistical analysis. To eliminate the impact of environmental factors, a linear mixing model was employed to calculate a BLUP value. In addition, using a ‘cor.test’ function in RStudio, coefficients of association and P values of the protein in different environments were calculated. A calculation method for broad-sense heritability is as follows (Knapp et al., 1983; Moran and Smith, 1918):

$${H}^{2} = \frac{σ {g}^{2}}{σ {g}^{2} + σ {ge}^{2} / e + σ {ε}^{2} / re} \times 100 \%$$

where σg² represents a genetic variance, σge² represents a variance caused by environment×genotype interactions, σε² represents a residual, e represents a location, and r represents the year.

### 1.3 Genotyping-by-Sequencing (GBS), Single Nucleotide Polymorphism (SNP) Extraction and Filtration

In the early growth of maize, leaf tissues were collected and subjected to freeze drying at −80° C., and a cetyltrimethylammonium bromide (CTAB) scheme was used for isolating and extracting genemic DNA (Poland et al., 2012). A GBS method (Zhou et al., 2016) was used for deep sequence processing of the DNA of 601 RILs and their parents, and genomic DNA digestion was performed using PstI and MspI. QIAquick PCR purification kit (QIAGEN, Valencia, CA, United States) was used for purifying PCR products. An Illumina NovaSeq 6000 platform (Illumina Inc., San Diego, CA, USA) was used for isolating and purifying fragments of 200-300 bp (including adapters and tags), followed by sequencing. Subsequently, original data were filtered to remove the adapters and low-quality sequences. Genome analysis toolkit software (McKenna et al., 2010) and maize B73 reference genome (Jiao et al., 2017) were used for SNP identification of the measured data. To ensure the quality of map, Plink v 1.9 (Purcell et al., 2007) was used for filtering out loci with a missing rate higher than 10% and SNPs with a minimum allele frequency (MAF) less than 5%. The parameters were set to --geno 0.2 and --maf 0.05 (SNP missing rate<20 and MAF<0.05).

### 1.4 PCA and Population Structure Analysis

Genome-wide complex trait analysis (GCTA, Yang et al., 2011) was used for performing PCA, and a scatterplot3d software package was used to visualize the results.

Admixture v1.3.0 was used for population structure analysis. First, K value was set for cross validation. It is believed that the K value with the lowest cross-validation error rate corresponds to the optimal number of subpopulations. Finally, a ggplot2 software package was used for visualizing the population structure.

### 1.5 LD Analysis

Nonrandom association between two or more genetic loci may be caused by factors such as historical recombination, selection pressure or population structure. R² value was used for measuring the degree of LD, ranging from 0 to 1, and a value closer to 1 indicates a higher degree of LD between two loci.

PopLDdecay (Zhang et al., 2019) software was used for calculating the degree of LD (r²) between makers, and a Plot_OnePop.pl software package was used for plotting an LD decay plot.

### 1.6 Map Construction and QTL Location

JoinMap4.0 software was used for constructing a genetic linkage map for pop2, with a logarithm of the odds (LOD) threshold of ≥5 set to determine a linkage group. The markers within the linkage group were ordered using a maximum likelihood method, and genetic distances between markers were calculated using a Kosambi function. Composite interval mapping (CIM) was employed to identify QTL locations of protein content in two populations in two environments. An LOD threshold of 2.5 was determined using 1000 random permutation tests (P<0.05). When a genetic distance between intervals exceeding the threshold line was less than 10 cM, they were considered as a single interval. A square of partial correlation coefficient (R²) was used to measure the degree of phenotypic variation explained (PVE) by individual QTLs. The QTL naming convention is as follows: q+P+chromosome serial number+detected QTL serial number, where q represents QTL, and P represents protein.

### 1.7 GWAS Analysis of Kernel Protein Content

A mixed linear model (MLM) in genome-wide efficient mixed model association (GEMMA) software was utilized to perform GWAS on the phenotypic mean and BLUP values of protein content, and the parameter was set to −1 mm 1 (Zhou et al., 2013). Population structure and genetic relationship were introduced as covariates to reduce errors (Yu et al., 2006). SNP loci meeting or exceeding the threshold were extracted using bedtools v1.7 (Strable et al., 2017), and significant SNPs were annotated using ANNOVAR software. The final results were visualized using CMplot v3.6.2 (Yin et al., 2021). A Manhattan plot was employed to display the distribution of markers, and a QQ plot was employed to assess the accuracy of the association analysis results.

### 1.8 Candidate Gene Prediction and Functional Annotation

Candidate genes were predicted within the significant SNP and its 20 kb range upstream and downstream. The candidate genes were predicted by reference to the maize B73 v4 reference genome sequence available in the MaizeGDB genome browser (https://www.maizegdb.org/). Functional annotations of the candidate genes were obtained using MaizeGDB and NCBI (https://www.ncbi.nlm.nih.gov/) databases.

### 1.9 Haplotype Analysis

Using Haploview v4.2 software, candidate genes related to protein content of seeds were detected in two environments. Initially, a haplotype map was constructed using high-density genome-wide SNPs. Subsequently, on the basis of the location information of significant loci related to a target trait and the LD analysis results, haplotypes where the significantly associated SNP loci were located were identified. Finally, genes within the haplotypes were annotated, to locate functionally associated genetic loci.

### 2. Results

### 2.1 Analysis of Kernel Protein Content

The protein phenotype data of the four subpopulations are statistically analyzed, as shown in Table 2 below:

TABLE 2
Statistical analysis of protein content

| | | | | | | | Coefficient | |
|---|---|---|---|---|---|---|---|---|
| | | | Standard | | | Range of | of variation | H² |
| Population | Environment | Mean | deviation | Skewness | Kurtosis | variation | (%) | (%) |
| pop1 | 22YS | 10.40 | 1.019 | −0.002 | −0.089 | 7.7-13.1 | 10.2 | 68.94 |
| | 23JH | 11.36 | 0.885 | 0.091 | −0.203 | 9.4-13.6 | 12.8 | |
| pop2 | 22YS | 10.38 | 0.987 | 0.325 | 0.728 | 8.0-13.8 | 10.5 | 74.73 |
| | 23JH | 11.06 | 0.875 | 0.566 | 0.184 | 9.4-13.8 | 12.6 | |
| pop3 | 22YS | 11.40 | 1.039 | 0.042 | 0.227 | 8.4-14.3 | 11.0 | 71.58 |
| | 23JH | 11.62 | 0.729 | 0.393 | 1.252 | 9.4-14.3 | 15.9 | |
| pop4 | 22YS | 10.76 | 1.006 | −0.132 | 0.337 | 7.7-13.3 | 10.7 | 56.64 |
| | 23JH | 11.04 | 0.892 | 0.399 | 1.337 | 8.1-14.0 | 12.4 | |

### 2.2 Structure Analysis

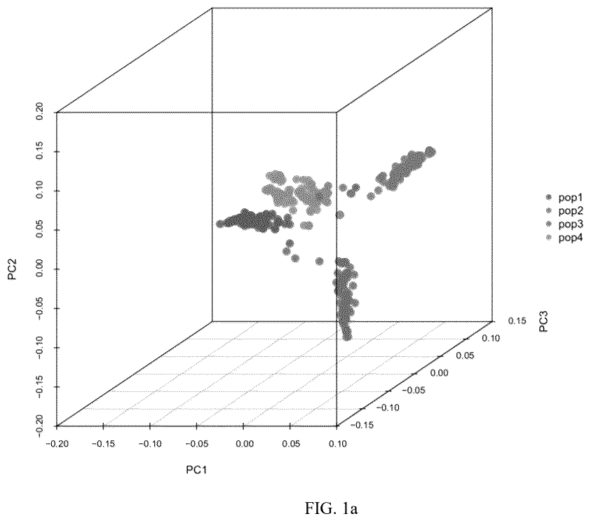

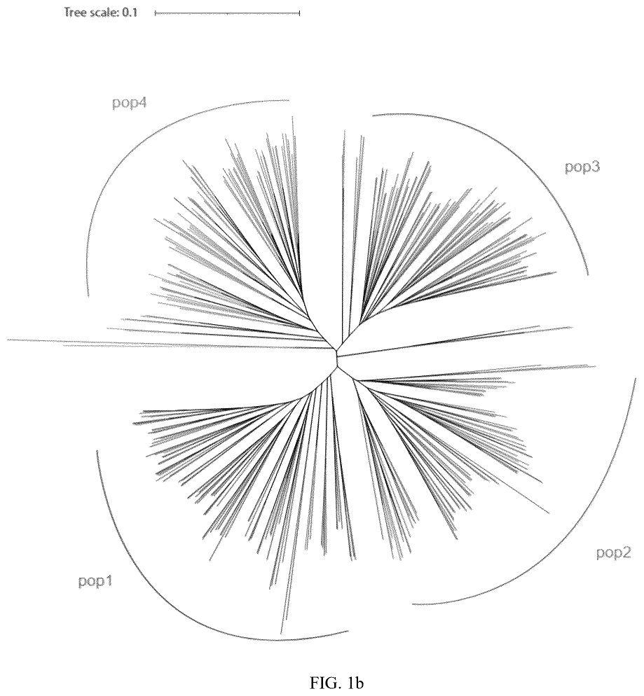

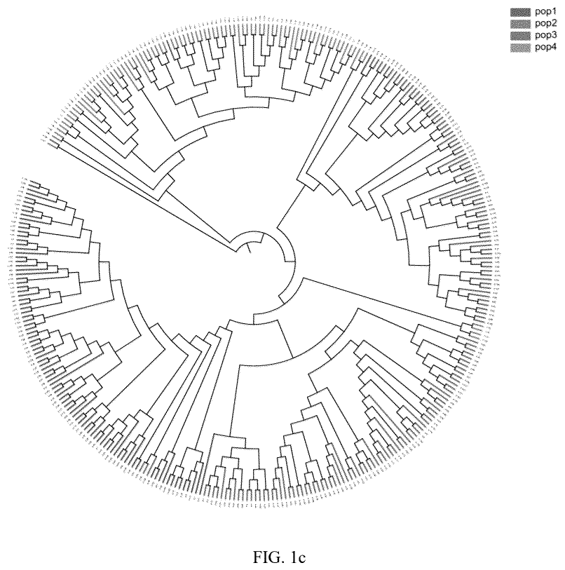

PCA results show that 601 RILs are divided into four subpopulations, which is consistent with the experimental design in this study (FIG. 1a). The phylogenetic tree shows that 601 RILs are mainly divided into four subpopulations, which is consistent with the PCA results (FIG. 1b and FIG. 1c). The population structure analysis indicates that when K=4, 601 RILs are divided into four subpopulations (FIG. 1d). The intermingling between populations may be caused by genetic drift or natural hybridization.

### 2.3 LD Decay Analysis

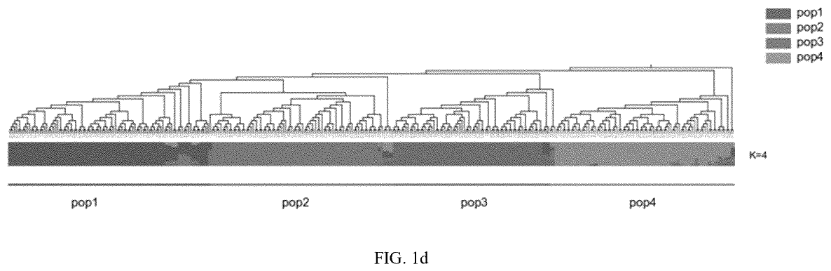

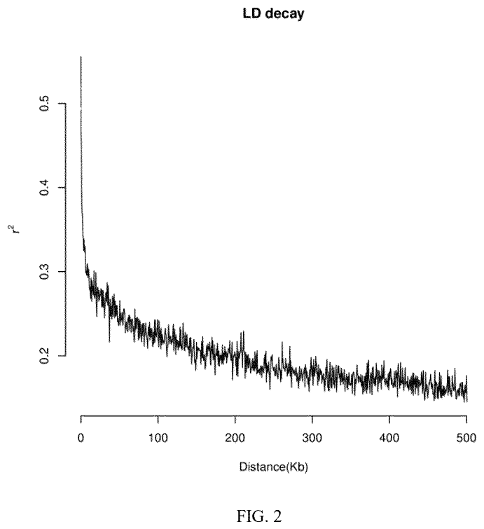

LD decay plot (FIG. 2) shows that with the increase of physical distance between loci, LD decays rapidly, indicating that LD decays faster. When a critical value of r² is 0.3, the physical distance of LD decay is estimated to be about 20 kb. Due to LD decaying at 20 kb, in this study, the 20 kb regions upstream and downstream of the significantly associated SNPs are screened to identify candidate genes.

### 2.4 Linkage Map and QTL Location

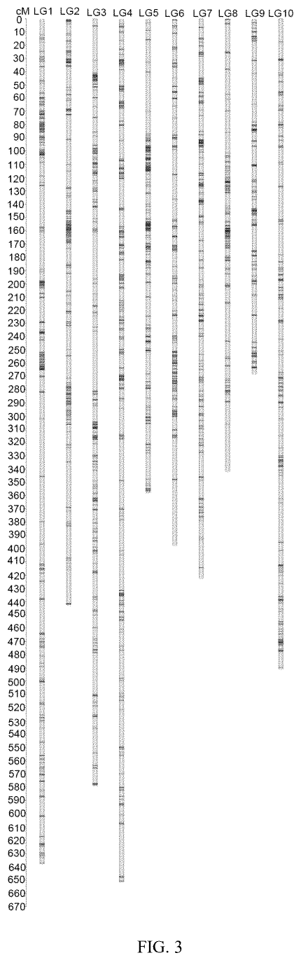

In this study, a high-density linkage map for pop2 is constructed for further QTL location of kernel protein content. The linkage map of pop2 is constructed using 1503 SNPs, with a total genetic distance of 4593.45 CM and a mean genetic distance between markers of 2.27 cM (FIG. 3).

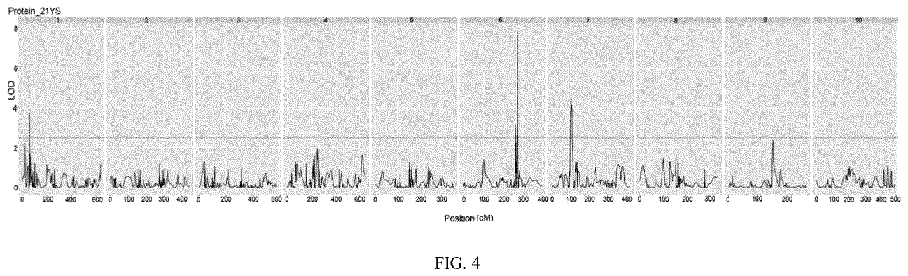

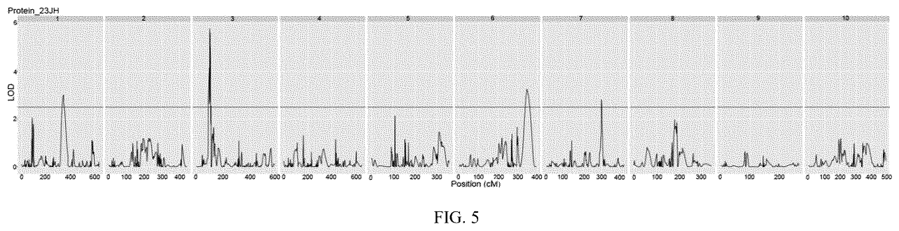

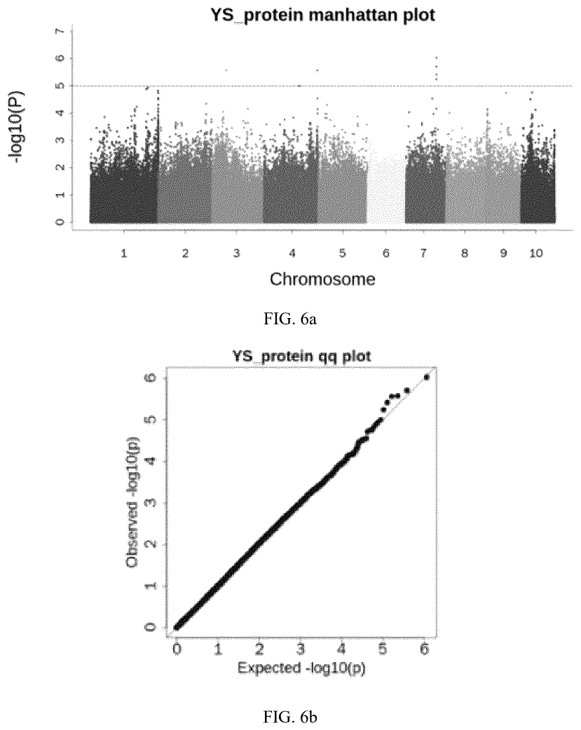

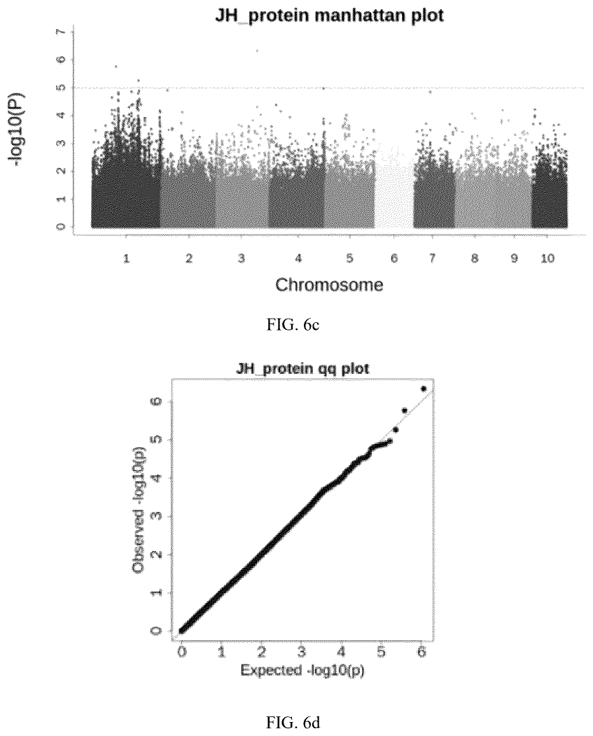

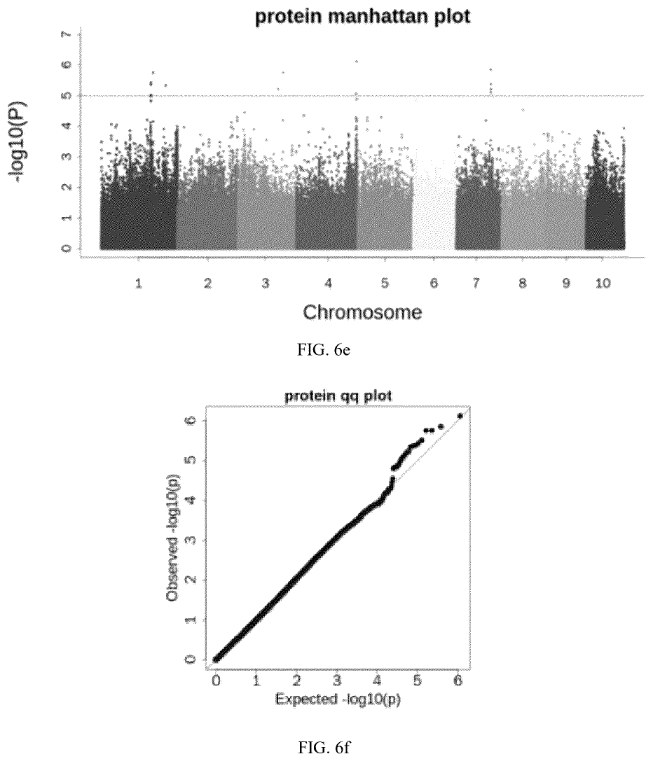

Significant QTLs of kernel protein content are screened on the basis of the linkage map, with an LOD threshold of 2.5. A total of eight significant QTLs associated with protein content are screened in pop2 (FIG. 4 and FIG. 5). Four significant QTLs identified at YS are located on chromosomes 1, 6, 7, and 10, respectively, with qP6-1 on chromosome 6 having the highest PVE of 26.78%. Four significant QTLs identified at JH are located on chromosomes 1, 3, 6, and 7 (Table 3).

TABLE 3
Significant QTL of protein

| | | | | Position | | Mapping | Additive | |
|---|---|---|---|---|---|---|---|---|
| Population | Environment | QTL | Chr | (cM) | LOD | interval | effect | R² (%) |
| | YS | qP1-1 | 1 | 60.33 | 3.82 | 103587284- | 1.08 | 11.36 |
| | | | | | | 273307800 | | |
| | | qP6-1 | 6 | 273.37 | 7.99 | 2799261- | −1.08 | 26.78 |
| | | | | | | 122662914 | | |
| | | qP7-1 | 7 | 103.74 | 4.71 | 102478219- | 0.47 | 16.25 |
| | | | | | | 102744609 | | |
| pop2 | | qP10-1 | 10 | 445.59 | 3.95 | 105437672- | 0.41 | 11.76 |
| | | | | | | 105516159 | | |
| | JH | qP1-2 | 1 | 344.02 | 3.00 | 107012609- | −0.28 | 10.79 |
| | | | | | | 307041717 | | |
| | | qP3-1 | 3 | 98.60 | 5.79 | 134076722- | −0.90 | 19.88 |
| | | | | | | 170719241 | | |
| | | qP6-2 | 6 | 347.64 | 3.25 | 141418854- | 0.30 | 10.36 |
| | | | | | | 174033170 | | |
| | | qP7-2 | 7 | 299.59 | 2.81 | 64905772- | −0.34 | 8.88 |
| | | | | | | 65850508 | | |

### 2.5 Integration of QTL Location and GWAS to Reveal Candidate Genes

Using a single research method to identify candidate genes may result in significant errors. Therefore, in this study, the QTL location results are integrated and compared with GWAS results, ultimately co-localizing seven candidate genes (Table 4). GWAS based on the mean phenotypic data shows that Zm00001d030087 screened at JH is co-localized with qP1-1 identified in pop2 at YS.

TABLE 4
QTL and GWAS co-localized protein candidate genes

| Candidate gene | Chr | QTL | SNP | Allele | Start ^{&} End | Annotation |
|---|---|---|---|---|---|---|
| Zm00001d030087 | 1 | qP1- | Chr1.P_{—} | G/A | 104645306- | ATPase_Vma12 |
| | | 1/2 | 104665306 | | 104685306 | |
| Zm00001d031730 | 1 | qP1- | Chr1.P_{—} | A/G | 199884924- | MATE_fam |
| | | 1/2 | 199904924 | | 199924924 | |
| Zm00001d033328 | 1 | qP1- | Chr1.P_{—} | C/T | 259156514- | Semialdehyde_{—} |
| | | 1/2 | 259176514 | | 259196514 | DH_NAD-bd |
| Zm00001d031989 | 1 | qP1- | Chr1.P_{—} | A/C | 208908509- | RRM_dom |
| | | 1/2 | 208928509 | | 208948509 | |
| Zm00001d031990 | | | | | | TMEM14 |
| Zm00001d031991 | | | | | | / |
| Zm00001d031992 | | | | | | / |

### 2.6 Haplotype Analysis

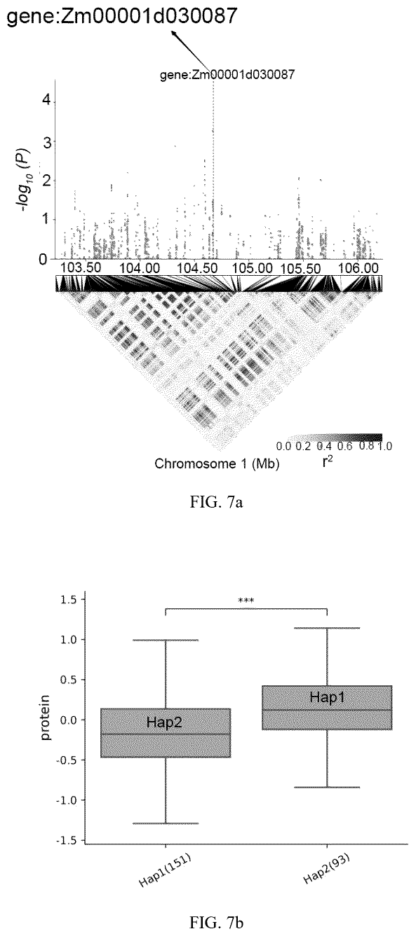

PVE analysis shows that the candidate gene Zm00001d030087 can explain 10.06% of the phenotypic variation in kernel protein content. Haplotype analysis shows that in the 601 RILs, Zm00001d030087 has two haplotypes, namely Hap1 and Hap2. The distribution frequency of Hap1 is 151 and the distribution frequency of Hap2 is 93. There is a significant difference between Hap1 and Hap2, and the kernel protein content of Hap2 is significantly higher than that of Hap1. Therefore, Hap2 is a dominant haplotype of Zm00001d030087 (FIG. 7a). The gene sequence of Zm00001d030087 is as shown in SEQ ID NO: 1:

ACAAACGGGATGGGTAACACTGCTGTAGGGTGATTCTCGAATCTCGAT
CTCCCTGCTCTACCCGCATCACCCCGCCGTTGCCGGCCCCTTCCTCCC
GCTTCCCACCTCGCCGTCGCCACCTCTTCCTCTTCCTCATCGCCGGCG
CTTGCGCGCCCCTTCTCCACCCCGTTGACCGCCGCACCCACGGCCTCG
CCGTTGCAGCACTCGTGCGATCCGGTCACGAGTGGAGCGCACGAGCTC
ATTCGCTGCTTCCGCCTCCCCCTCTTCCTTGCCGGAGCCCGCGCCTCC
TCTCCTCGCCGGCGCCTTCGCCTGCTCCTGCGCCCCCTTCTCCACGTC
GCACCCACGGCCTCGTGTTGCTGCCCTCGTGAGATCCGGTCGCGAGCG
CCCGACCTAGGCCGCTGCCTCAGTCTCTCCTCGCTCGGTCGCATCACC
ACCGCCGGAGACGGACCGAGGTGCTCTGCGAGACTCCGCGCCATAGAG
CTCCTCCTACGGCGGGTTCTTTTCCGTCCGGTAGCTGGGTTCCCTGAA
GCCAGTCATGGCGATGACCACCGCCGTGACGGGCCTCGCCGTCGCAGC
AACCGCTCCCATTCGCTCCTTCCTCTCGTCCGCCGCCGCCTCTGTCAG
CCTGCCCGCCGATCTTCGTGACCTCGCCTCCGACCTAGCCTCGGACCC
TGCCGTCTCCTACCGCTCTCTGCGTGCCATCTGGTGCGCCACCTCGCC
GGACACCCGCCCGCCGCTCCGCGACCTCCTCCAGGGCGCCGACTTCGT
GCTACCCAGCCCGAAACCTCGTGAGAAGGTTCTTTCTCTCAGTCTTTG
GTGGCACTGTCTGTCCCGGCCTGTGCGTACCGGCAAATAGGTGGGTTT
TGATTCTGATTCGGTTTAGTCTGTCATGTGGTTGGTTTGCAGAGCGAT
GAGCTGAAGGCGAGGCTGGAGAAGCTCCGGGAGACACAGGAGAGGAAG
GAGTATGCTGAGCTCGTCAGGGACGTCGCTCCGCCCAGCAAGGATGAC
GCTCCTGAGCCCTTTTCATCCTACAAGGATCAGATAGGATTTGGTACT
ACCCAACCGCCCTATTTTCAAATTGCTCTATGCAATCTTCCTACTGCT
TGTTTCATGGATTACACTTGTTCCATTGAAGTAATAGGAATATAGGAT
GACACCGTGTGTGTATTTCAACATAATTGGAGCAATATAGTTCAAAAG
CTATAACATACGAGTTGTGGAGCAATATCTTCTCTCTTCATCAATTGT
TCCAAATGCAGAGGTAGGATCTACTATTTTTTAAAACACACACACATA
AAGACAAGAATAAACAAATAAGATAATGTAAAATAAATTTGAGAATAA
TATTTTTGTAAATACCACTCGAACAAAGATCAATCTTCCGTGTGCTCG
TATCTAAAGCAATGATGCCATTAGAATTTAGACCTGAGGTGCAGCACA
TGAAAATTGTTCTACCTCCACCACTACATTAGTGTATACGTCCAAGAG
GCATTTATCTAGATTTAAAGTGAACAACGAACTACTAAAAATCTAGTT
TCAGGATTTGTTTTTTTGAACTATCAACTAAATCAGGTTATAATAACC
TGAGTTTAATCTTTAATCAAGATTACATTAATAACTTGGTGGTGTGAA
AATCTATTTCTCGCTCAAAGGTCTGAAGTTCAGAGGCCATGTATTGTG
TTGTTTTTCTTCTTTATTTTTAAAGTTATTATTACTTTACCGGTTTCT
ATTTTTATATTTATATTGAACGTACGTGACAAAAAACAAAATTTAATC
TAACCGGAAATATAAAAAAATGAAATCGTGAAAAGTCATAGAAAAAGA
GAAGGAACACGATACATCTACCTGACCGGCCAGCAGCCATCGCTGCGC
AGTGTGCACTTGTGTCCGGCAGCACAAGCCCGCATCTGGCTGCCTCCG
CGTGCACCCACGCCTGGACGCCCGTCGCCCGCTGCACCAGGCGGCCCG
GGGCCTGGATCTACCCGAGCTTTCCTGGGCTCAGTCTTTGGATTTGAA
AAACTATCTTGAAGACTCATGTATAAGCATGAATGATTGCCAATAGAA
GAATTCTTCTTTTCAGAATTAGAAACTTTAAATTTTGATGCGGTCTGT
GACTCTGTGCTACCCTACTTGGTGCAAAAGCATTTTGGTTATGTACTA
CTCCCTCCGTTCTTTTTTATTTGTCGCGATCTAGTTCAAAAATGAATT
AGCGGGCGACAAATATTCGAGAACGGAGGTAGTATATAATAGCTAGAG
GCCAAAGTTTGATGGATCTATTTAATTTGTGCACCATGTTTACTTTTT
GTTTGTTGTTGGCTTCAGCGTGTACTGTAATTCCTCTATCTTGTTTGG
GAATCTGGGGATTTTAAGGGAAAACAGACTCCTTAAGTTTAAATTAGT
GTTGGTGAAACTTGTAATCAAGTTTTCAGGATGGAGTAACCATGCATG
CCAATAGTAGTGAAATGATACAAATGTTTCTTCACTTAAATGCACTAC
TACACAAACCTGCAATGCGCAATTAAGAATGATTGCTATGATGCGAGA
ATTGTTCTTTTACGAATTCAGAACTTTTTAACTCCAAGAAGTTTAGCC
AAGTACTGGTCTTGTATGGTAATGTCAGTAAGAACGAGTGGGATCTAA
CATTTCTGTGCTTATTTGCAATGGGAGTAGAGTTATACAATAAATCCC
TCGTTTCATTTTTAACTATAGCTAGCAAAAATGGTTTATGTTTTTTTT
GTTAACGTGTAAAAATTGGTGTTTATGAGTCTTCATAAATTCAAAAAA
ATAAGTTGAAACATGGATGCTAGATTCAGGTTTGAACTTGTTTTTGTT
GCAACTAATCGAGCATTTTGGTTATCTACAATGTAATTGACTGAAGCT
GAACCGCTAAAGCTTGTTGGATCTACTTTAATTTCTGCATTTCCTTTA
TTTTCTTTCTAATTGTGGTTATTACTGGGAACTATAATTCCTTTACCT
TTGCTAATGGGACCCTAGGGTCTTGTGCAAAAAGGATTTCTCAATTAA
ACAGGTGCATGTGAAACTTGCAATCAAGTATCCAGGAAGTGTTAATAG
ACTAGTGGTAAATGAAAGCCTACAAAATTTGTACAGTTATGCATGCCA
TCGGTAATGACATCATACAAATACGTTTTTGACTGAAAATTTACTGCT
AGACAAACCCACGGTGCACAATCAGGAAATAGTTTGTCACTTCAAGTT
AATGGTGGCTTTTTCATCTGAATGCTTTGGCCTCTAGAATCACTCTTC
GCGTATCCATAATCACCAGTCACTTTCATTAAAAGGGGTGATTTTCTA
TCTAATTCTGCTATGCACCTTTTTAAGTAAAATGATATTAGGTGATCT
TTTGTTTTGGTTATATCGATCCAATTTATCATCAATATTAAATCCACA
TGCCTCTAAAATAGAGGCAAACTTTTCTATGTACAAAGTGATCGATAG
TGTTCTTTTCCTATAAGTATATTTATCCTCTTAAGATGACATACAGTC
TATATCTGAGCTGAGGCTGCTAGGATTTTCTTGTATGTTTTGTATGGT
TTTCATTGTTCAATTGACTTTTCTTCTTTTTCAGGTCTGCATGTCGTG
GTTATTATGTTCACAGGTTACTTGGTAGGATTTGCTATGTTCAAAGCT
CTGTTCAACAATAATGCTGTACTGGTAAGTTCATTAGGCCACTTCTTG
TAGGGATGTAGTATCTTGTACATTGAATGGGAAAGCCAAATAATTTTG
CCTTCCTCTGTAGCTGCTAATTTGTTTGCCATTCCTGATAAGATTAAA
CCTCACAGAGTTAACCTTGTCATGGCTTTTTAGAACGCTGCTGGAGGT
ATCTTAGGATTAGTTGGTGGCATGCTGGTTGAGACCATTCTCTTCATC
ATCAGATCATCGAGTAAAGAGTTAGCCTCTGTTCCAAGATCAAAGAAA
GCTCAGTAGACTCTTGAAATGCTATCATCAGTTAGTGGTGAGAGCTAT
AAGTGGCCTTAGCTTGTGGAAGTCTATGGAGCATTGGTATGGGTTTGA
AAATGCAATTCATCGAATATATGCAATTGACATCCGATTTGAATTTGC
ATCATGAAGGAATAGGGCAGAGGGATTTGATTTCAATATTATTAGCTA
CCCAGAATTTTGTATCTCGCCCAATGATTGGCTCGATGATAATTGAAG
AACATGTACCTACGGATATGTAAAAAGTACATACAAGAAGATGGTATG
CTTAACAGCTTAAGTACCATTGGAGAGTATATTGGCTTGAAATTTTGC
AGTTAAAACTTAAAAGTAAGTTGTTATGTTTGCACGTGAGGTTTTGTT
TGTGAATAAGCATATTTTATCCTAAGATTTTTCTCAGATTTCTTTTTT
ATCATAGCATTTTGAGTAAATCGAAATGAATTTTTTGTTGGTATATGC
ACGATTGTTTAGAGCAACTCCAATAGTTACGTAAATTTTAGCTCTCTA
AATAATAGATTTAAGAAGTTGCTAAATGGCTTTTGGAGTAAAAAATGC
GAGTTATCCAATAGTTTTCTAAATATAGGTTGTAATTTTGTTTTGTAT
CTATCCATATAAAAAAATAAGTCAAAAAAACTACATAATGCAGTCAAC
ATTTTTGTTTAGAGAGTTGTTAAATGGTTGCCAAATGTAGAGAGAAAA
TGAGGTTAGATGACAACTTGTTAAATTTAGAAAGTTCATTTAAAGAAC
TGTTGGAGAATAATTTTTATATTAACTACCTACATTATTGATTTAGTA
AGTCTTTTAGAGAACTACTGGAGTTGCTCTTATTTAGCTAGTTAAAAG
GATTAAGACACTTTAGAGCATCTTCAATAGTTATGTAAATTTAGCACC
CTAAGAGCAACTCCAGTAGTTCTCTAAAAGACTTCCTAAATCAATAAT
TTAGGTAGTTAACATGAAAACTATTCTCCAACAGTTCCCTAAATTAAC
TTTCTAAATTTAACAACTTGTCATCTAACCTCATTTTCTCTCTACATT
TGGCAACCATTTAACAACTCCCTAAACAAAAATGTTGACTGCATTGTA
TAGTTTTTGTGACTTATTTTTTATGTGGATAAATACAAAACAAAATTA
CAACCTATATTTAGAGAACTATTGGAGAACTCACATTTATTTACTCCA
AAAGCCATTTAGCAACTTCTTAAATATGTGATTTAGAGAGCTAAAATT
TACATAACTATTGGAGTTGCTCTAAATTATAGATTTAAGAAGTTGCTA
AATCATTTTAAGGAGTAGAAAAATGTGTGAGCTCCAACAGTTCTCTAT
TTTATTAGTTACTAATTTTTAGAATTATCTACATCAACAAATAAAGTG
AGCATGTTTTCTATTTTTAATTGAGTTAGCACATCAATAACCTTTGAA
AATGTCTTGAAAGGTCGAAACAAAATATCTGGAAAGGTCGAAGTTATC
GATGGAAGATCAGAATGTCGCCGCGGCGACGACATGAAGACAACACAT
GGCACTGTAGATAGATTTGTGGGATGACGCAAAACAGTGGCCGAAATA
TGGTTTTCGGGATGGATTTAGAACCAACATTGATTTAGGGAGTCCCAA
CGAGGTGGTAAATATAGGGATGAAATTGGATTTTGTATGGAGTTTGTA
AATTTAAGGATCTGCTTTATATAACTGTTGGAAAAGAGTTTTCCCAAA
AAA.

The embodiment described above is merely used for illustrating the technical solutions of the disclosure, rather than limiting the disclosure. Although the disclosure is described in detail by reference to the foregoing embodiment, it is to be understood by those ordinary skilled in the art that the technical solutions in each embodiment can still be modified or some technical features can be replaced equivalently, and those modifications or replacements cannot make the essence of the corresponding technical solutions out of the spirit and scope of the technical solutions in each embodiment of the disclosure.

1. A method for identifying a gene associated with protein content of maize kernels, the gene being Zm00001d030087 gene as shown in SEQ ID NO: 1, the method comprising:
i. constructing a multi-parent population (MPP) to generate recombinant inbred lines (RILs), wherein parents differ in kernel protein content;
ii. measuring kernel protein content of the RILs in at least two environments:
iii. performing genotyping-by-sequencing (GBS) on the RILs;
iv. conducting quantitative trait locus (QTL) mapping to construct a genetic linkage map for a subpopulation;
v. performing genome-wide association study (GWAS) to select SNPs associated with protein content at p<0.01;
vi. co-localizing QTL intervals from step iv with the SNPs from step v to identify candidate genes within 20 kb regions; and
validating the candidate genes by haplotype analysis.


SEQUENCE LISTING (1 sequence)

SEQ ID NO: 1 - DNA, 5667 bp, unidentified
  source 1..5667: mol_type = other DNA
acaaacgggatgggtaacactgctgtagggtgattctcgaatctcgatctccctgctctacccgcatcaccccgccgttgccggccccttcctcccgcttcccacctcgccgtcgccacctcttcctcttcctcatcgccggcgcttgcgcgccccttctccaccccgttgaccgccgcacccacggcctcgccgttgcagcactcgtgcgatccggtcacgagtggagcgcacgagctcattcgctgcttccgcctccccctcttccttgccggagcccgcgcctcctctcctcgccggcgccttcgcctgctcctgcgcccccttctccacgtcgcacccacggcctcgtgttgctgccctcgtgagatccggtcgcgagcgcccgacctaggccgctgcctcagtctctcctcgctcggtcgcatcaccaccgccggagacggaccgaggtgctctgcgagactccgcgccatagagctcctcctacggcgggttcttttccgtccggtagctgggttccctgaagccagtcatggcgatgaccaccgccgtgacgggcctcgccgtcgcagcaaccgctcccattcgctccttcctctcgtccgccgccgcctctgtcagcctgcccgccgatcttcgtgacctcgcctccgacctagcctcggaccctgccgtctcctaccgctctctgcgtgccatctggtgcgccacctcgccggacacccgcccgccgctccgcgacctcctccagggcgccgacttcgtgctacccagcccgaaacctcgtgagaaggttctttctctcagtctttggtggcactgtctgtcccggcctgtgcgtaccggcaaataggtgggttttgattctgattcggtttagtctgtcatgtggttggtttgcagagcgatgagctgaaggcgaggctggagaagctccgggagacacaggagaggaaggagtatgctgagctcgtcagggacgtcgctccgcccagcaaggatgacgctcctgagcccttttcatcctacaaggatcagataggatttggtactacccaaccgccctattttcaaattgctctatgcaatcttcctactgcttgtttcatggattacacttgttccattgaagtaataggaatataggatgacaccgtgtgtgtatttcaacataattggagcaatatagttcaaaagctataacatacgagttgtggagcaatatcttctctcttcatcaattgttccaaatgcagaggtaggatctactattttttaaaacacacacacataaagacaagaataaacaaataagataatgtaaaataaatttgagaataatatttttgtaaataccactcgaacaaagatcaatcttccgtgtgctcgtatctaaagcaatgatgccattagaatttagacctgaggtgcagcacatgaaaattgttctacctccaccactacattagtgtatacgtccaagaggcatttatctagatttaaagtgaacaacgaactactaaaaatctagtttcaggatttgtttttttgaactatcaactaaatcaggttataataacctgagtttaatctttaatcaagattacattaataacttggtggtgtgaaaatctatttctcgctcaaaggtctgaagttcagaggccatgtattgtgttgtttttcttctttatttttaaagttattattactttaccggtttctatttttatatttatattgaacgtacgtgacaaaaaacaaaatttaatctaaccggaaatataaaaaaatgaaatcgtgaaaagtcatagaaaaagagaaggaacacgatacatctacctgaccggccagcagccatcgctgcgcagtgtgcacttgtgtccggcagcacaagcccgcatctggctgcctccgcgtgcacccacgcctggacgcccgtcgcccgctgcaccaggcggcccggggcctggatctacccgagctttcctgggctcagtctttggatttgaaaaactatcttgaagactcatgtataagcatgaatgattgccaatagaagaattcttcttttcagaattagaaactttaaattttgatgcggtctgtgactctgtgctaccctacttggtgcaaaagcattttggttatgtactactccctccgttcttttttatttgtcgcgatctagttcaaaaatgaattagcgggcgacaaatattcgagaacggaggtagtatataatagctagaggccaaagtttgatggatctatttaatttgtgcaccatgtttactttttgtttgttgttggcttcagcgtgtactgtaattcctctatcttgtttgggaatctggggattttaagggaaaacagactccttaagtttaaattagtgttggtgaaacttgtaatcaagttttcaggatggagtaaccatgcatgccaatagtagtgaaatgatacaaatgtttcttcacttaaatgcactactacacaaacctgcaatgcgcaattaagaatgattgctatgatgcgagaattgttcttttacgaattcagaactttttaactccaagaagtttagccaagtactggtcttgtatggtaatgtcagtaagaacgagtgggatctaacatttctgtgcttatttgcaatgggagtagagttatacaataaatccctcgtttcatttttaactatagctagcaaaaatggtttatgttttttttgttaacgtgtaaaaattggtgtttatgagtcttcataaattcaaaaaaataagttgaaacatggatgctagattcaggtttgaacttgtttttgttgcaactaatcgagcattttggttatctacaatgtaattgactgaagctgaaccgctaaagcttgttggatctactttaatttctgcatttcctttattttctttctaattgtggttattactgggaactataattcctttacctttgctaatgggaccctagggtcttgtgcaaaaaggatttctcaattaaacaggtgcatgtgaaacttgcaatcaagtatccaggaagtgttaatagactagtggtaaatgaaagcctacaaaatttgtacagttatgcatgccatcggtaatgacatcatacaaatacgtttttgactgaaaatttactgctagacaaacccacggtgcacaatcaggaaatagtttgtcacttcaagttaatggtggctttttcatctgaatgctttggcctctagaatcactcttcgcgtatccataatcaccagtcactttcattaaaaggggtgattttctatctaattctgctatgcacctttttaagtaaaatgatattaggtgatcttttgttttggttatatcgatccaatttatcatcaatattaaatccacatgcctctaaaatagaggcaaacttttctatgtacaaagtgatcgatagtgttcttttcctataagtatatttatcctcttaagatgacatacagtctatatctgagctgaggctgctaggattttcttgtatgttttgtatggttttcattgttcaattgacttttcttctttttcaggtctgcatgtcgtggttattatgttcacaggttacttggtaggatttgctatgttcaaagctctgttcaacaataatgctgtactggtaagttcattaggccacttcttgtagggatgtagtatcttgtacattgaatgggaaagccaaataattttgccttcctctgtagctgctaatttgtttgccattcctgataagattaaacctcacagagttaaccttgtcatggctttttagaacgctgctggaggtatcttaggattagttggtggcatgctggttgagaccattctcttcatcatcagatcatcgagtaaagagttagcctctgttccaagatcaaagaaagctcagtagactcttgaaatgctatcatcagttagtggtgagagctataagtggccttagcttgtggaagtctatggagcattggtatgggtttgaaaatgcaattcatcgaatatatgcaattgacatccgatttgaatttgcatcatgaaggaatagggcagagggatttgatttcaatattattagctacccagaattttgtatctcgcccaatgattggctcgatgataattgaagaacatgtacctacggatatgtaaaaagtacatacaagaagatggtatgcttaacagcttaagtaccattggagagtatattggcttgaaattttgcagttaaaacttaaaagtaagttgttatgtttgcacgtgaggttttgtttgtgaataagcatattttatcctaagatttttctcagatttcttttttatcatagcattttgagtaaatcgaaatgaattttttgttggtatatgcacgattgtttagagcaactccaatagttacgtaaattttagctctctaaataatagatttaagaagttgctaaatggcttttggagtaaaaaatgcgagttatccaatagttttctaaatataggttgtaattttgttttgtatctatccatataaaaaaataagtcaaaaaaactacataatgcagtcaacatttttgtttagagagttgttaaatggttgccaaatgtagagagaaaatgaggttagatgacaacttgttaaatttagaaagttcatttaaagaactgttggagaataatttttatattaactacctacattattgatttagtaagtcttttagagaactactggagttgctcttatttagctagttaaaaggattaagacactttagagcatcttcaatagttatgtaaatttagcaccctaagagcaactccagtagttctctaaaagacttcctaaatcaataatttaggtagttaacatgaaaactattctccaacagttccctaaattaactttctaaatttaacaacttgtcatctaacctcattttctctctacatttggcaaccatttaacaactccctaaacaaaaatgttgactgcattgtatagtttttgtgacttattttttatgtggataaatacaaaacaaaattacaacctatatttagagaactattggagaactcacatttatttactccaaaagccatttagcaacttcttaaatatgtgatttagagagctaaaatttacataactattggagttgctctaaattatagatttaagaagttgctaaatcattttaaggagtagaaaaatgtgtgagctccaacagttctctattttattagttactaatttttagaattatctacatcaacaaataaagtgagcatgttttctatttttaattgagttagcacatcaataacctttgaaaatgtcttgaaaggtcgaaacaaaatatctggaaaggtcgaagttatcgatggaagatcagaatgtcgccgcggcgacgacatgaagacaacacatggcactgtagatagatttgtgggatgacgcaaaacagtggccgaaatatggttttcgggatggatttagaaccaacattgatttagggagtcccaacgaggtggtaaatatagggatgaaattggattttgtatggagtttgtaaatttaaggatctgctttatataactgttggaaaagagttttcccaaaaaa